(19) United States
(12) Patent Application Publication
(10) Pub. No.: US 2025/0152266 A1
(43) Pub. Date: May 15, 2025

(54) System And Method For Generating A Patient-Specific Milling Path

(71) Applicant: MAKO Surgical Corp., Weston, FL (US)

(72) Inventors: Hans-Ulrich Becker, Muellheim (DE); Mohammad Javad Barakchi Fard, Weston, FL (US); Matthew Thompson, Woodbridge, CT (US); Gregory Garcia, Parkland, FL (US); Rene Schoene, Glottertal (DE); Garrett Thomas Joyal, Gillette, NJ (US)

(73) Assignee: MAKO Surgical Corp., Weston, FL (US)

(21) Appl. No.: 18/940,482

(22) Filed: Nov. 7, 2024

Related U.S. Application Data
(60) Provisional application No. 63/547,902, filed on Nov. 9, 2023.

(51) Int. Cl. A61B 34/30; A61B 17/16; A61B 34/10

(52) U.S. Cl. CPC A61B 34/30; A61B 17/1615; A61B 2017/1602; A61B 2034/105; A61B 2034/107

(57) ABSTRACT

A computer-implemented method is provided. The computer-implemented method generates a milling path for a tool of a surgical system, the milling path designed to enable the tool to resect material from a bone, the method including obtaining a model of the bone, intersecting an allowed volume with the model for defining a resection volume intended to be removed from the bone, and generating a plurality of sections. The method also includes, for a section, identifying a sub-volume of the resection volume corresponding to the section; generating milling path segments designed to enable the tool to remove the sub-volume of the resection volume; identifying, for the sub-volume of the resection volume, a region to be avoided by the tool; generating transition path segments designed to avoid the region; and generating the milling path by combining the milling path segments and the transition path segments.

### CROSS-REFERENCE TO RELATED APPLICATIONS

The subject application claims priority to and all the benefits of U.S. Provisional Patent Application No. 63/547,902, filed Nov. 9, 2023, the entire contents of the aforementioned application being hereby incorporated by reference.

### BACKGROUND

Surgical procedures require the use of specialized tools to perform tasks requiring a high degree of accuracy and precision. Such surgical procedures require precise positioning of tools and/or implants relative to a patient's anatomy. Within the field of orthopedic procedures, the removal of tissue to create cavities, for example, within a bone rely on great care by the surgeon to successfully accomplish the procedure. For example, it can be especially challenging to remove hard tissue, such as bone, to create a cavity without impacting soft tissue adjacent to the hard tissue.

There exists a need in the art to remove tissue to create cavities that overcomes one or more of the problems set forth above.

### SUMMARY

According to one aspect, a computer-implemented method is provided. The computer-implemented method generates a milling path for a tool of a surgical system, the milling path designed to enable the tool to resect material from a bone that defines a socket for a joint, the computer-implemented method comprising: obtaining a model of the bone including the socket; intersecting an allowed volume with the model of the socket for defining a resection volume intended to be removed from the bone; generating a plurality of sections; for at least one section: identifying a sub-volume of the resection volume corresponding to the section; generating one or more milling path segments designed to enable the tool to remove the sub-volume of the resection volume; identifying, for the sub-volume of the resection volume corresponding to the section, a region to be avoided by the tool; generating one or more transition path segments designed to avoid the region; and generating the milling path by combining the one or more milling path segments and the one or more transition path segments.

According to a second aspect, a non-transitory computer readable medium comprising instructions executable by one or more processors is provided. The instructions implement a software program for generating a milling path for a tool of a surgical system, the milling path designed to enable the tool to resect material from a bone that defines a socket for a joint, the software program being configured to: obtain a model of the bone including the socket; intersect an allowed volume with the model of the socket to define a resection volume intended to be removed from the bone; generate a plurality of sections; for at least one section: identify a sub-volume of the resection volume corresponding to the section; generate one or more milling path segments designed to enable the tool to remove the sub-volume of the resection volume; identify, for the sub-volume of the resection volume, a region to be avoided by the tool; generate one or more transition path segments designed to avoid the region; and generate the milling path by combining the one or more milling path segments and the one or more transition path segments.

According to a third aspect, a surgical system is provided. The surgical system comprises a manipulator comprising a robotic arm formed of a plurality of links and joints and supporting a tool; a control system configured to generate a milling path designed to enable the tool to resect material from a bone that defines a socket for a joint, wherein to generate the milling path, the control system is configured to: obtain a model of the bone including the socket; intersect an allowed volume with the model of the socket to define a resection volume intended to be removed from the bone; generate a plurality of sections; for at least one section: identify a sub-volume of the resection volume corresponding to the section; generate one or more milling path segments designed to enable the tool to remove the sub-volume of the resection volume; identify, for the sub-volume of the resection volume, a region to be avoided by the tool; and generate one or more transition path segments designed to avoid the region; and generate the milling path by combining the one or more milling path segments and the one or more transition path segments; wherein the control system is configured to control the manipulator to move the tool along the generated milling path.

According to a fourth aspect, a computer-implemented method for generating a milling path for a tool of a surgical system is provided. The milling path is designed to enable the tool to resect material from a bone that defines a socket for a joint, the computer-implemented method comprising: obtaining a model of the bone including the socket; intersecting an allowed volume with the model of the socket for defining a resection volume intended to be removed from the bone, wherein the allowed volume defines a boundary for the tool; and generating a first section and a second section, the first section being adjacent to the second section; wherein a distance between the first section and the second section is based on a scallop height.

According to a fifth aspect, a computer-implemented method for generating a milling path for a tool of a surgical system is provided. The milling path designed to enable the tool to resect material from a bone that defines a socket for a joint, the computer-implemented method comprising: obtaining a model of the bone including the socket; intersecting an allowed volume with the model of the socket for defining a resection volume intended to be removed from the bone, wherein the allowed volume defines a boundary for the tool; defining a safeguard volume disposed in the allowed volume, wherein the safeguard volume defines a boundary for the tool, and wherein a distance is defined between the boundary of the safeguard volume and the boundary of the allowed volume; generating a plurality of sections; for the plurality of sections, identifying a sub-volume of the resection volume and generating a milling path designed to enable the tool to remove the sub-volume of the resection volume; and generating a plurality of transition path segments for connecting the milling paths of adjacent sections, wherein the plurality of transition path segments extend along the boundary of the safeguard volume between adjacent sections and form a spiral.

According to a sixth aspect, a computer-implemented method for generating a milling path for a tool of a system, the milling path designed to enable the tool to resect material, the computer-implemented method comprising: obtaining a model of a material for resection; intersecting an allowed volume with the model of the material for defining a resection volume intended to be removed; generating a plurality of sections; for at least one section: identifying a sub-volume of the resection volume corresponding to the section; generating one or more milling path segments designed to enable the tool to remove the sub-volume of the resection volume; identifying, for the sub-volume of the resection volume, a region to be avoided by the tool; generating one or more transition path segments designed to avoid the region; and generating the milling path by combining the one or more milling path segments and the one or more transition path segments.

Also provided are a non-transitory computer readable medium (or computer program product) configured to implement any of the above aspects. Also provided are a surgical system comprising a controller configured to implement any of the above aspects.

Any of the above aspects may be combined in part or in whole. Any one or more of the above aspects may be combined in part or in whole with any one or more of the implementations below.

In some implementations, the computer-implemented method further comprises generating a safeguard volume within the allowed volume, wherein the safeguard volume defines a boundary for the tool, wherein the allowed volume defines a boundary for the tool, wherein the boundary of the safeguard volume is spaced apart from the boundary of the allowed volume, and wherein the one or more transition path segments are defined by a geometry of the safeguard volume. In some implementations, the computer-implemented method further comprises generating an inner allowed volume within the allowed volume, the inner allowed volume defining a boundary for the tool, wherein the boundary of the inner allowed volume is spaced apart from the boundary of the allowed volume, and wherein the safeguard volume is defined within the inner allowed volume and the boundary of the safeguard volume is spaced apart from the inner allowed volume. In some implementations, the tool includes a spherical cutting burr having a burr radius, and wherein the boundary of the inner allowed volume is spaced apart from the boundary of the allowed volume by the burr radius. In some implementations, the allowed volume extends along an axis. In some implementations, the allowed volume, the inner allowed volume, and the safeguard volume are coaxial about the axis. In some implementations, the allowed volume, the inner allowed volume, and the safeguard volume are each rotationally symmetric. In some implementations, a geometry of the allowed volume is based on a geometry of an implant to be inserted into the socket. In some implementations, the allowed volume includes a cylindrical portion including a first end and a second end along the axis, the first end and the second end defining a height of the cylindrical portion. In some implementations, the allowed volume includes a spherical dome portion having a center located on the axis, and wherein the spherical dome portion is integrated with the cylindrical portion and extends from the second end of the cylindrical portion.

In some implementations, the computer-implemented method further comprises identifying that the sub-volume of the resection volume is interrupted by a gap defining an absence of the resection volume, wherein the region to be avoided by the tool includes the gap. In some implementations, the computer-implemented method further comprises determining whether a dimension of the gap is greater than a threshold value and generating the one or more transition path segments in response to determining that the dimension of the gap is greater than the threshold value.

In some implementations, the section is further defined as a sector extending radially from the axis toward the boundary of the allowed volume. In some implementations, the sector is defined as being normal to the boundary of the allowed volume. In some implementations, the section is further defined as a first sector, wherein a section adjacent to the first section is further defined as a second sector, and wherein identifying the sub-volume of the resection volume corresponding to the first sector comprises identifying the sub-volume of the resection volume between the first and second sectors.

In some implementations, the one or more milling path segments are generated based on an intersection of the inner allowed volume and the section. In some implementations, generating the one or more transition path segments includes generating one or more transition path segments to connect the one or more milling path segments for the at least one section, and wherein the one or more transition path segments extend along the section between the one or more milling path segments and the boundary of the safeguard volume. In some implementations, generating a connecting transition path segment for connecting a milling path segment of a first section and a milling path segment of an adjacent second section, wherein the connecting transition path segment extends along the boundary of the safeguard volume between the first section and the adjacent second section. In some implementations, the plurality of connecting transition path segments form a spiral extending along the boundary of the safeguard volume.

In some implementations, the bone is a pelvis or a scapula.

### BRIEF DESCRIPTION OF THE DRAWINGS

Advantages of the present disclosure will be readily appreciated, as the same becomes better understood by reference to the following detailed description, when considered in connection with the accompanying drawings. Non-limiting and non-exhaustive embodiments of the present disclosure are described with reference to the following figures, wherein like numerals refer to like parts throughout the various views unless otherwise specified.

FIG. 1 is a perspective view of a system for manipulating an anatomy of a patient.

FIG. 2 is a block diagram of a controller that may be used with the system shown in FIG. 1.

FIG. 3 is a flow chart of a method of generating a milling path for a tool of the system shown in FIG. 1.

FIG. 4 is a perspective view of a model of a bone.

FIG. 5 is a perspective view of a boundary volume generated by the method shown in FIG. 3.

FIG. 6 is a diagrammatic view of a milling path generated by the method shown in FIG. 3.

FIG. 7A is a perspective view of the model shown in FIG. 4 being intersected with the boundary volume shown in FIG. 5.

FIG. 7B is a perspective view of a resection volume for resection by a milling path generated by the method shown in FIG. 3.

FIG. 8A is a diagrammatic view of a plurality of reference planes, wherein a first and second section correspond to a first and second reference plane.

FIG. 8B is a diagrammatic view of a plurality of points, wherein a first and second section correspond to a first and second point.

FIGS. 9A and 9B are diagrammatic views of a first and second section, respectively, intersecting the resection volume shown in FIG. 7B.

FIG. 10A is a diagrammatic view of a plurality of reference planes separated by a step distance.

FIGS. 10B and 10C are diagrammatic views of the plurality of references planes of FIG. 10A, wherein a step distance separating the reference planes is calculated based on preserving a desired scallop height.

FIG. 11A is a diagrammatic view of a first and second sector generated by the method shown in FIG. 3.

FIG. 11B is a perspective view of a first and second sector generated by the method shown in FIG. 3 and a resection sub-volume.

FIGS. 12A and 12B are diagrammatic views of a milling path for resecting the resection sub-volume of FIG. 11B.

FIG. 13 is a diagrammatic view of a milling path segment generated by the method shown in FIG. 3 for resecting a resection sub-volume.

FIGS. 14A and 14B are diagrammatic views of a transition path segment for avoiding resecting soft tissue.

FIGS. 15 and 16 are diagrammatic views of alternative transition path segments.

FIG. 17 is a diagrammatic view of a milling path resecting a resection volume.

FIGS. 18A and 18B are perspective views of connecting transition path segments.

### DETAILED DESCRIPTION

In the following description, numerous specific details are set forth in order to provide a thorough understanding of the present invention. It will be apparent, however, to one having ordinary skill in the art that the specific details need not be employed and/or not be employed exactly as described to practice the present invention. In some instances, well-known materials or methods have not been described in detail in order to avoid obscuring the present invention.

### I. System Overview

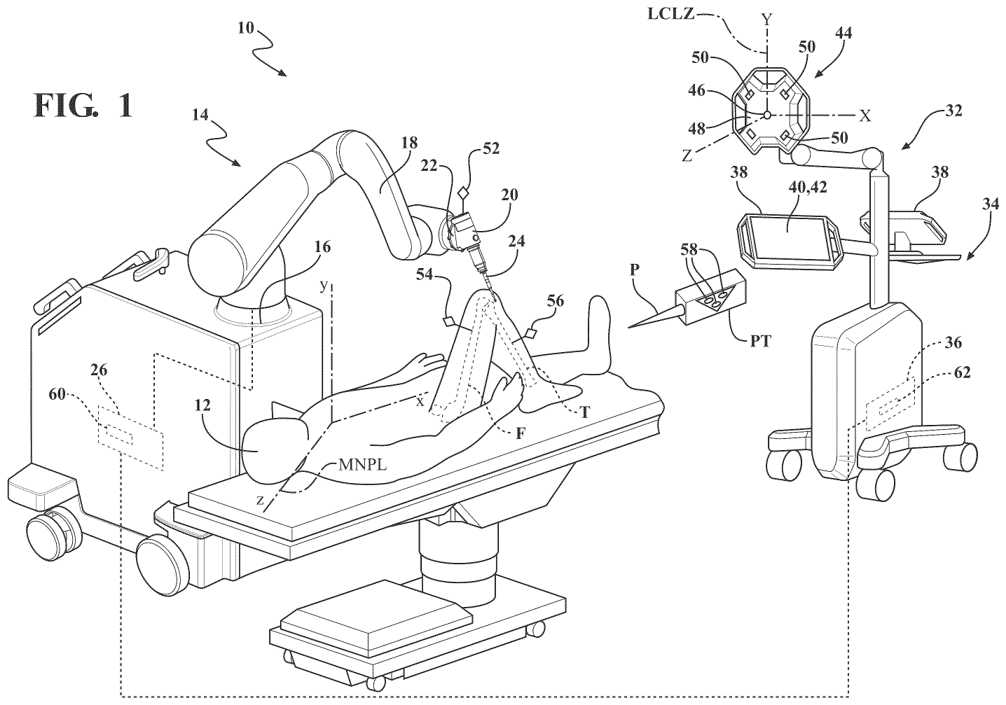

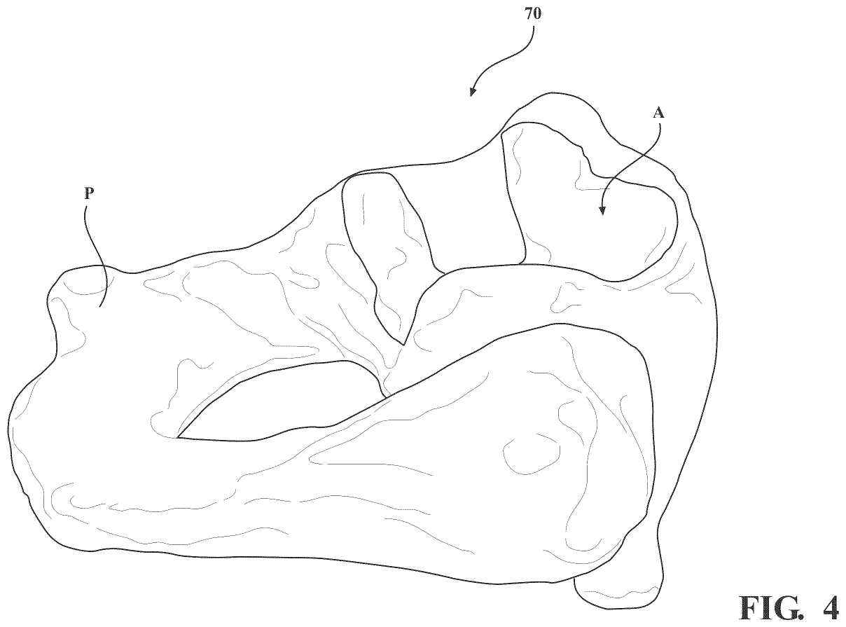

FIG. 1 is a perspective view of a system 10 for manipulating an anatomy of a patient 12. More specifically, system 10 is a robotic surgical cutting system for cutting away material from the anatomy of the patient 12, such as bone or soft tissue. In FIG. 1, the patient 12 is undergoing a surgical procedure. The anatomy in FIG. 1 includes a femur (F) and a tibia (T) of the patient 12. Referring to FIG. 4, the anatomy also includes a pelvis P and an acetabulum A. The surgical procedure may involve tissue removal and may also involve the insertion of one or more implants or grafts (e.g., bone or cartilage grafts, real or artificial ligaments, etc.) into a portion of the patient's anatomy. In some embodiments, the surgical procedure involves partial or total knee or hip replacement surgery. Some of the types of implants that may be used in the surgical procedure are shown in U.S. Pat. No. 9,381,085, entitled, “Prosthetic Implant and Method of Implantation,” the disclosure of which is hereby incorporated by reference. Those skilled in the art appreciate that the system and method disclosed herein may be used to perform other procedures, surgical or non-surgical, or may be used in industrial applications or other applications where robotic systems are utilized.

The system 10 includes a surgical robotic manipulator 14. The manipulator 14 has a base 16 and a linkage 18. The linkage 18 may comprise links forming a serial arm or parallel arm configuration. A tool, such as an end effector 20, removably couples to the manipulator 14 and is movable relative to the base 16 to interact with the surgical environment, and more specifically, the anatomy. The end effector 20 is grasped by the operator. One exemplary arrangement of the manipulator 14 and the end effector 20 is described in U.S. Pat. No. 9,119,655, entitled, “Surgical Manipulator Capable of Controlling a Surgical Instrument in Multiple Modes,” the disclosure of which is hereby incorporated by reference. The manipulator 14 and the end effector 20 may be arranged in alternative configurations. The end effector 20 includes an energy applicator 24 designed to contact the tissue of the patient 12 at the surgical site. The end effector 20 may have various configurations depending on the application. The energy applicator 24 may be a cavity creation tool, such as a drill, a saw blade, a bur, an ultrasonic vibrating tip, a probe, a stylus, a reamer, a rasp, impactor, or the like. The manipulator 14 also houses a manipulator computer 26, or other type of control unit. The end effector 20 can be like that shown in U.S. Pat. No. 9,566,121, entitled, “End Effector of a Surgical Robotic Manipulator,” which is hereby incorporated by reference.

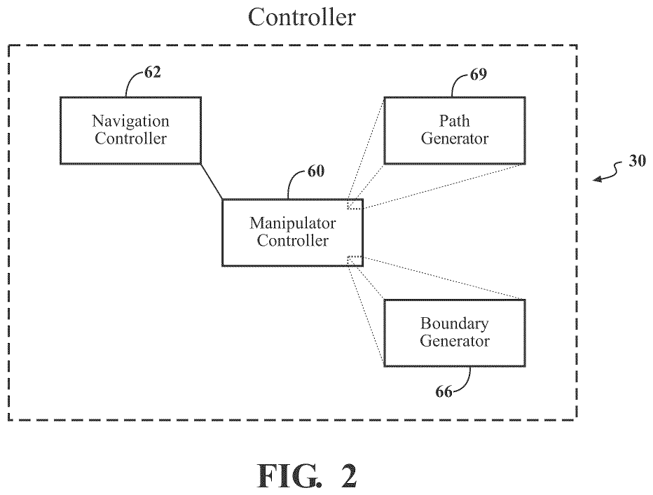

Referring to FIG. 2, the system 10 includes a controller 30. The controller 30 includes software and/or hardware for controlling the manipulator 14. The controller 30 directs the motion of the manipulator 14 and controls an orientation of the end effector 20 with respect to a coordinate system. In one embodiment, the coordinate system is a manipulator coordinate system MNPL (see FIG. 1). The manipulator coordinate system MNPL has an origin, and the origin is located at a point on the manipulator 14. One example of the manipulator coordinate system MNPL is described in U.S. Pat. No. 9,119,655, entitled, “Surgical Manipulator Capable of Controlling a Surgical Instrument in Multiple Modes,” the disclosure of which is hereby incorporated by reference.

The system 10 further includes a navigation system 32. One example of the navigation system 32 and components related thereto is described in U.S. Pat. No. 9,008,757, filed on Sep. 24, 2013, entitled, “Navigation System Including Optical and Non-Optical Sensors,” hereby incorporated by reference. The navigation system 32 is set up to track movement of various objects. Such objects include, for example, the end effector 20, and the anatomy or portions thereof, e.g., femur F, tibia T, and acetabulum A. The navigation system 32 tracks these objects to gather position information of each object in a localizer coordinate system LCLZ. Coordinates in the localizer coordinate system LCLZ may be transformed to the manipulator coordinate system MNPL using conventional transformation techniques. The navigation system 32 is also capable of displaying a virtual representation of their relative positions and orientations to the operator.

The navigation system 32 includes a computer cart assembly 34 that houses a navigation computer 36, and/or other types of control units. A navigation interface is in operative communication with the navigation computer 36. The navigation interface includes one or more displays 38. First and second input devices 40, 42 such as a keyboard and mouse or touch screen may be used to input information into the navigation computer 36 or otherwise select/control certain characteristics of the navigation computer 36. Other input devices 40, 42 are contemplated including voice-activation. The controller 30 may be implemented on any suitable device or devices in the system 10, including, but not limited to, the manipulator computer 26, the navigation computer 36, and any combination thereof.

The navigation system 32 also includes a localizer 44 that communicates with the navigation computer 36. In one embodiment, the localizer 44 is an optical localizer and includes a camera unit 46. The camera unit 46 has an outer casing 48 that houses one or more optical position sensors 50. The system 10 includes one or more trackers. The trackers may include a pointer tracker PT, a tool tracker 52, a first patient tracker 54, and a second patient tracker 56. The trackers include markers 58. The markers 58 may be light emitting diodes or LEDs. In other embodiments, the markers 58 are passive markers, such as reflectors, which reflect light emitted from the camera unit 46. Those skilled in the art appreciate that other suitable tracking systems and methods not specifically described herein may be utilized, such as electromagnetic localization systems, ultrasound, and the like.

In the illustrated embodiment of FIG. 1, the first patient tracker 54 is firmly affixed to the femur F of the patient 12 and the second patient tracker 56 is firmly affixed to the tibia T of the patient 12 for use in a knee replacement surgery, for example. Alternatively, the first patient tracker 54 may be affixed to the femur F of the patient 12 and the second patient tracker 56 may be affixed to an acetabulum A or pelvis P of the patient 12 for use in a hip replacement surgery. The patient trackers 54, 56 are firmly affixed to sections of bone. The tool tracker 52 is firmly attached to the end effector 20. It should be appreciated that the trackers 52, 54, 56 may be fixed to their respective components in any suitable manner.

The trackers 52, 54, 56 communicate with the camera unit 46 to provide position data to the camera unit 46. The camera unit 46 provides the position data of the trackers 52, 54, 56 to the navigation computer 36. In one embodiment in which trackers 54 and 56 are coupled to the femur F and acetabulum A of the patient, the navigation computer 36 determines and communicates position data of the femur F and acetabulum A and position data of the end effector 20 to the manipulator computer 26. Alternatively, the navigation computer 36 may determine position data of the tibia T or another portion of the anatomy to which tracker 56 may be coupled and may communicate the position data to the manipulator computer 26. Position data for the femur F, acetabulum A, and end effector 20 may be determined by the tracker position data using conventional registration/navigation techniques. The position data includes position information corresponding to the position and/or orientation of the femur F, acetabulum A, end effector 20 and any other objects being tracked. The position data described herein may be position data, orientation data, or a combination of position data and orientation data.

The manipulator computer 26 transforms the position data from the localizer coordinate system LCLZ into the manipulator coordinate system MNPL by determining a transformation matrix using the navigation-based data for the end effector 20 and encoder-based position data for the end effector 20. Encoders (not shown) located at joints of the manipulator 14 are used to determine the encoder-based position data. The manipulator computer 26 uses the encoders to calculate an encoder-based position and orientation of the end effector 20 in the manipulator coordinate system MNPL. Since the position and orientation of the end effector 20 are also known in the localizer coordinate system LCLZ, the transformation matrix may be generated.

### II. Software Module Overview

As shown in FIG. 2, the controller 30 further includes software modules. The software modules may be part of a computer program or programs that operate on the manipulator computer 26, navigation computer 36, or a combination thereof, to process data to assist with control of the system 10. The software modules include sets of instructions stored in memory on the manipulator computer 26, navigation computer 36, or a combination thereof, to be executed by one or more processors of the computers 26, 36. Additionally, software modules for prompting and/or communicating with the operator may form part of the program or programs and may include instructions stored in memory on the manipulator computer 26, navigation computer 36, or a combination thereof. The operator interacts with the first and second input devices 40, 42 and the one or more displays 38 to communicate with the software modules.

As shown in FIG. 2, the controller 30 may include a manipulator controller 60. The manipulator controller 60 may process data to direct motion of the manipulator 14. The manipulator controller 60 may receive and process data from a single source or multiple sources.

The controller 30 may include a navigation controller 62 for communicating the position data relating to the femur F, acetabulum A (or other portions of the anatomy such as the tibia T), and end effector 20 to the manipulator controller 60. The manipulator controller 60 may receive and process the position data provided by the navigation controller 62 to direct movement of the manipulator 14. In one embodiment, as shown in FIG. 1, the navigation controller 62 is implemented on the navigation computer 36.

The manipulator controller 60 or navigation controller 62 may also communicate positions of the patient 12 and end effector 20 to the operator by displaying an image of the anatomy (e.g., acetabulum A and/or femur F) and the end effector 20 on the display 38. The manipulator computer 26 or navigation computer 36 may also display instructions or request information on the display 38 such that the operator may interact with the manipulator computer 26 for directing the manipulator 14.

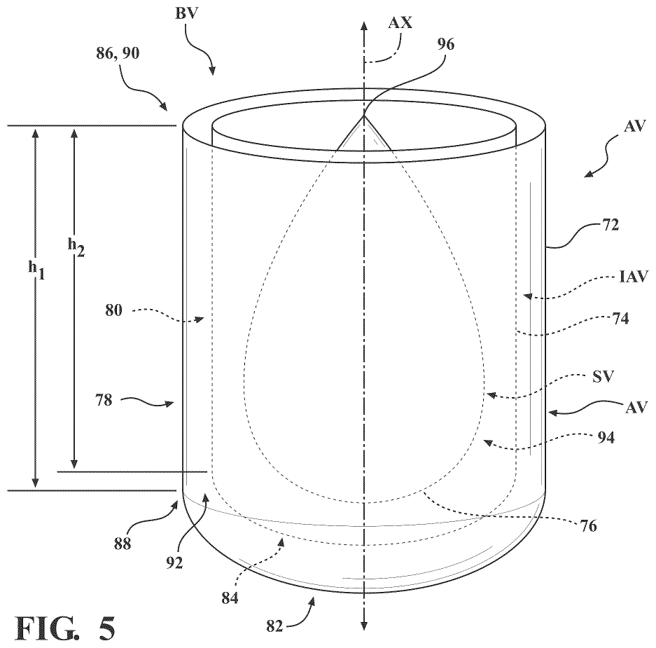

As shown in FIG. 2, the controller 30 includes a boundary generator 66. The boundary generator 66 may generate a boundary of the end effector 20. For example, referring to FIG. 5, the boundary generator 66 may generate a boundary volume BV. The boundary volume BV may include an allowed volume AV, an inner allowed volume IAV, and a safeguard volume SV. The allowed volume AV, the inner allowed volume IAV, and the safeguard volume SV each define a boundary of the end effector 20, to be described in greater detail below. The boundary generator 66 is a software module that may be implemented on the manipulator controller 60, as shown in FIG. 2. Alternatively, the boundary generator 66 may be implemented on other components, such as the navigation controller 62.

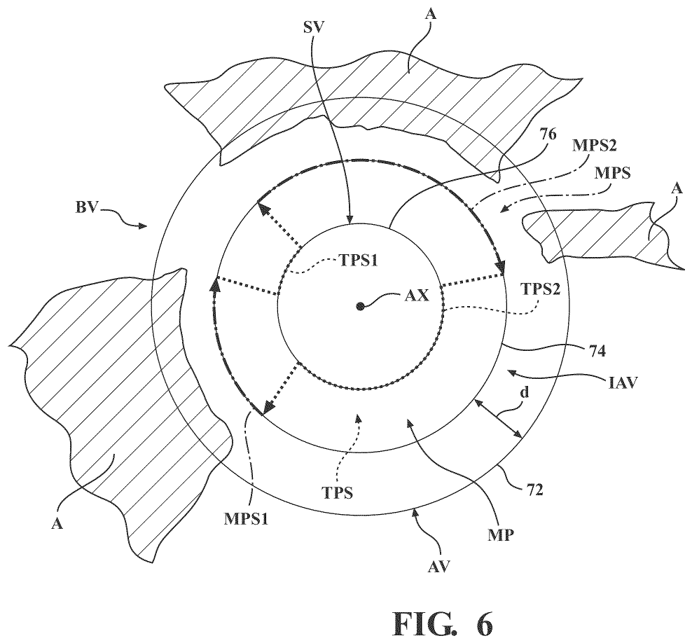

As shown in FIG. 2, the controller 30 includes a tool path generator 69. Referring to FIG. 6, the tool path generator 69 may be configured to generate a milling path MP for the end effector 20 to move along. The tool path generator 69 is a software module that may be implemented on the manipulator controller 60, as shown in FIG. 2. Alternatively, the tool path generator 69 may be implemented on other components, such as the navigation controller 62.

### III. Generating Patient-Specific Milling Path

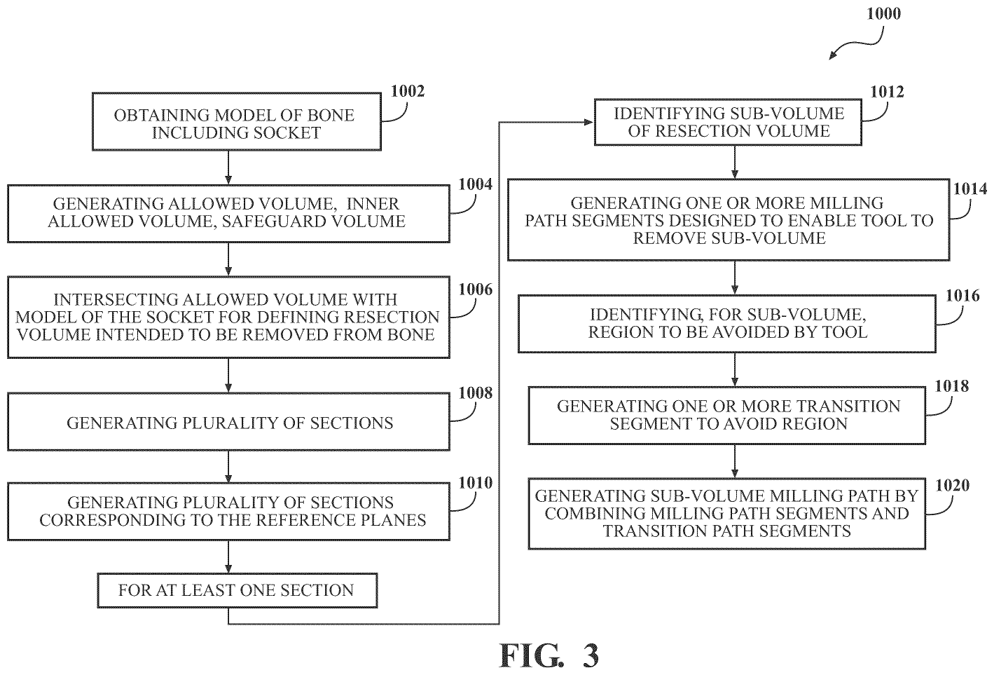

FIG. 3 illustrates a method 1000 of generating a patient-specific milling path MP for the end effector 20, the milling path MP being designed to enable the end effector 20 to resect material from a bone that defines a socket for a joint. In the instances illustrated herein, the method 1000 generates the milling path MP for total hip replacement surgery. An example milling path MP is shown in FIG. 6, where the milling path MP is designed as a path that a center of the end effector 20 moves along to enable the end effector 20 to resect material from a pelvis P (the bone) that defines an acetabulum A (the socket) for a hip joint. More particularly, when the end effector 20 travels along the milling path MP, the end effector 20 resects material from the acetabulum A to prepare the acetabulum A for placement of an implant therein.

In other instances, the method 1000 may generate the milling path MP for other types of surgery. For example, the method 1000 may generate the milling path MP for shoulder replacement surgery. Specifically, the milling path MP may be generated to resect material from a scapula defining a glenoid for a shoulder joint. In another example, the method 1000 may generate the milling path MP for knee replacement surgery.

The method 1000, as described herein, may be performed pre-operatively, or during an operation. For example, the method 1000 may be executed before a procedure such that the method 1000 generates the milling path MP before a surgery is performed. The method 1000 may also be executed while a surgery is being performed such that the milling path MP may be generated on-the-fly.

i. Obtaining Model of Hard Tissue

As shown in FIG. 3, the method 1000 includes a step 1002 of obtaining a model 70 of hard tissue, such as the model 70 shown in FIG. 4. Any suitable software module of the controller 30 may perform step 1002. For example, either the manipulator controller 60 or the navigation controller 62 may obtain the model 70 of hard tissue. The model 70 may be obtained using any suitable imaging modality. For example, the model 70 may be obtained via MRI, CT scan, ultrasound, x-ray, etc.

Generally, the model 70 indicates hard tissue to be resected by the end effector 20. For example, the model 70 may be a model 70 of a bone including a socket. In the instance of FIG. 4 the model 70 includes a pelvis P defining an acetabulum A.

Soft tissue may be omitted from such models 70. For example, soft tissue may exist in areas where the model 70 does not indicate the presence of hard tissue. As such, the method 1000 generates the milling path MP to resect the hard tissue illustrated by the model 70 and to avoid soft tissue. Referring to FIG. 6, the milling path MP may include milling path segments MPS and transition segments TS. As will explained in greater detail below, the end effector 20 moves along the milling path segments MPS to resect material from the acetabulum A; and the end effector 20 moves along the transition segments TS to avoid resection of soft tissue.

Additionally, the model 70 may be a patient-specific model representing the hard tissue of a patient to be operated on. As follows, the milling path MP generated to resect the hard tissue illustrated by the model 70 may be considered a patient-specific milling path MP.

ii. Generating Boundary Volume

Referring to FIG. 3, the method 1000 also includes a step 1004 of generating the allowed volume AV, the inner allowed volume IAV, and the safeguard volume SV of the boundary volume BV. The allowed volume AV, the inner allowed volume IAV, and the safeguard volume SV are shown in FIG. 5. As previously stated, the boundary generator 66 may perform step 1004 and generate the allowed volume AV, the inner allowed volume IAV, and the safeguard volume SV.

Referring to FIG. 5, the inner allowed volume IAV and the safeguard volume SV are generated within the allowed volume AV. More specifically, the safeguard volume SV is generated within the inner allowed volume IAV, which is generated within the allowed volume AV. While the inner allowed volume IAV and the safeguard volume SV are generated within the allowed volume AV, the inner allowed volume IAV and the safeguard volume SV may not be entirely encapsulated by the allowed volume AV. Similarly, while the safeguard volume SV is generated within the inner allowed volume IAV, the safeguard volume SV may not be entirely encapsulated by the inner allowed volume IAV.

The allowed volume AV extends along an axis AX. In the instance of FIG. 5, the allowed volume AV, the inner allowed volume IAV, and the safeguard volume SV each extend along the axis AX and are coaxial about the axis AX. In other instances, the allowed volume AV, the inner allowed volume IAV, and the safeguard volume SV may not be coaxial. For example, the allowed volume AV, the inner allowed volume IAV, and the safeguard volume SV may each extend along different axes. Additionally, in the instance of FIG. 5, the allowed volume AV, the inner allowed volume IAV, and the safeguard volume SV are each rotationally symmetric about the axis AX. In other instances, the allowed volume AV, the inner allowed volume IAV, and the safeguard volume SV may be rotationally asymmetric.

The allowed volume AV, the inner allowed volume IAV, and the safeguard volume SV each define a boundary for the end effector 20. As shown in FIG. 6, the allowed volume AV defines a boundary 72 for a surface of the end effector 20 such that, when the end effector 20 travels along the milling path MP, a surface of the end effector 20 does not exceed the boundary 72 of the allowed volume AV. The inner allowed volume IAV and the safeguard volume SV each define a boundary 74, 76, respectively, for the center of the end effector 20. As shown in FIG. 6, boundaries 74, 76 are defined such that, when the end effector 20 travels along a transition segment TS, the center of the end effector 20 is confined between the boundaries 74, 76 such that the center of the end effector 20 does not exceed the boundaries 74, 76.

As shown in FIG. 6, the boundary 74 of the inner allowed volume IAV is spaced apart from the boundary 72 of the allowed volume AV. Specifically, the boundary 74 of the inner allowed volume IAV is spaced apart from the boundary 72 of the allowed volume AV by a distance d. In instances where the end effector 20 includes a spherical cutting burr having a burr radius, the distance d may correspond to the burr radius. For instance, the distance d may be equivalent to the burr radius. In such instances, when the end effector 20 travels along a milling path segment MPS, the center of the end effector 20 is confined to the boundary 74 of the inner allowed volume IAV such that the surface of the end effector 20 does not exceed the boundary 72 of the allowed volume AV and resects material from the acetabulum A that is within the boundary 72 of the allowed volume AV.

As shown in FIG. 6, the boundary 76 of the safeguard volume SV is spaced apart from the boundary 72 of the allowed volume AV. The safeguard volume SV may be spaced apart from the boundary 72 of the allowed volume AV such that, when the end effector 20 travels along a transition path segment TPS, the center of the end effector 20 is disposed between the inner allowed volume IAV and the safeguard volume SV to avoid resecting soft tissue.

The allowed volume AV, the inner allowed volume IAV, and the safeguard volume SV may include any suitable shape. For example, a geometry of the allowed volume AV and/or the inner allowed volume IAV may be based on a geometry of an implant to be inserted into a socket of a bone, such as the acetabulum A of the pelvis P. As another example, a geometry of the safeguard volume SV may be based on a volume where soft tissue typically does not exist within a socket of a bone. For instance, the geometry of the safeguard volume SV may be based on a volume within the acetabulum A where soft tissue typically does not exist.

In the instance of FIG. 5, each of the allowed volume AV and the inner volume IAV include a cylindrical volume and spherical volume. Specifically, the allowed volume AV and the inner volume IAV include cylindrical portion 78, 80 and a spherical dome portion 82, 84. As shown in FIG. 5, the cylindrical portion 78 of the allowed volume AV includes a first end 86 and a second end 88 along the axis AX, with the first end and the second end defining a height h₁ of the cylindrical portion 78. In the instance of FIG. 5, the spherical dome portion 82 of the allowed volume AV is a hemisphere. Also shown, the cylindrical portion 80 of the inner allowed volume IAV includes a first end 90 and a second end 92 along the axis AX, with the first end and the second end defining a height h₂ of the cylindrical portion 80. In the instance of FIG. 5, the spherical dome portion 84 of the inner allowed volume IAV is a hemisphere.

In other instances, the allowed volume AV and the inner allowed volume IAV may include alternate geometries. For example, the allowed volume AV and the inner allowed volume IAV may include a teardrop-shaped volume and/or a conical volume. In one such example, the allowed volume AV and the inner allowed volume IAV may each include a conical portion between respective cylindrical portions 78, 80 and spherical dome portions 82, 84. Furthermore, in other instances, the allowed volume AV and the inner allowed volume IAV may include differing geometries. For example, the allowed volume AV may include a teardrop-shaped volume and the inner allowed volume IAV may include only a cylindrical volume.

In the instance of FIG. 5, the safeguard volume SV includes a teardrop-shaped volume. As shown, the teardrop-shaped volume extends along the axis AX, with the safeguard volume SV including a globular portion 94 tapering to a point 96. In other instances, the safeguard volume SV may include alternate geometries. For example, the safeguard volume SV may include a conical volume, a spherical volume, and/or a cylindrical volume.

iii. Defining Resection Volume to be Removed

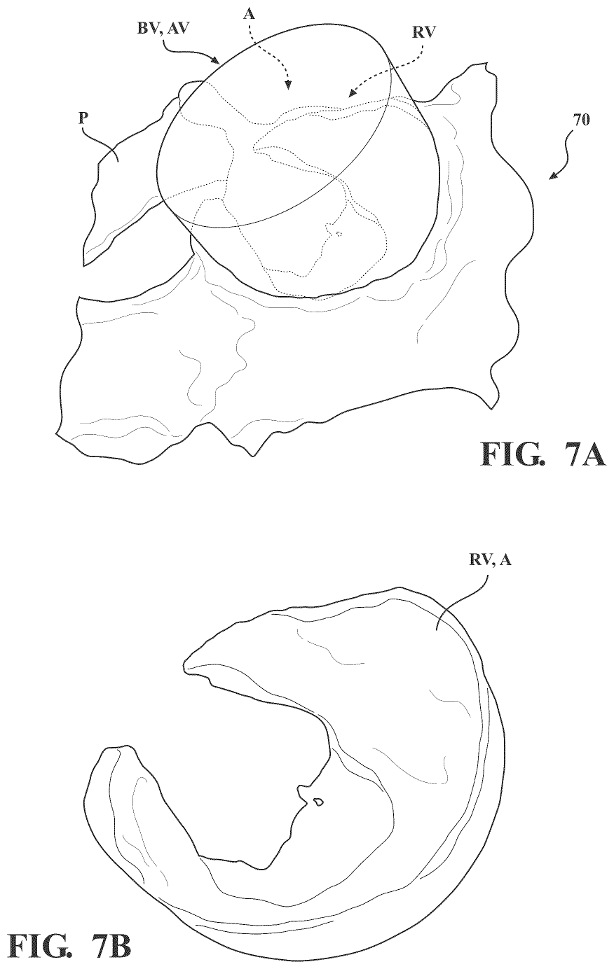

Referring to FIG. 3, the method 1000 includes a step 1006 of intersecting the allowed volume AV with the model 70 of the socket for defining a resection volume intended to be removed from the bone with the milling path MP. An instance of step 1006 is illustrated in FIGS. 7A and 7B. As shown, the allowed volume AV is intersected with the model 70 of the acetabulum A for defining a resection volume RV to be removed from the pelvis P in FIG. 7A. The resection volume RV is further shown in FIG. 7B.

As stated above, soft tissue may be omitted from the models 70. As such, the resection volume RV typically corresponds to hard tissue intended to be removed by the end effector 20. In the instances of FIGS. 7A and 7B, the resection volume RV corresponds to material to be removed from the acetabulum A.

iv. Generating Plurality of Reference Planes Intersecting Resection Volume

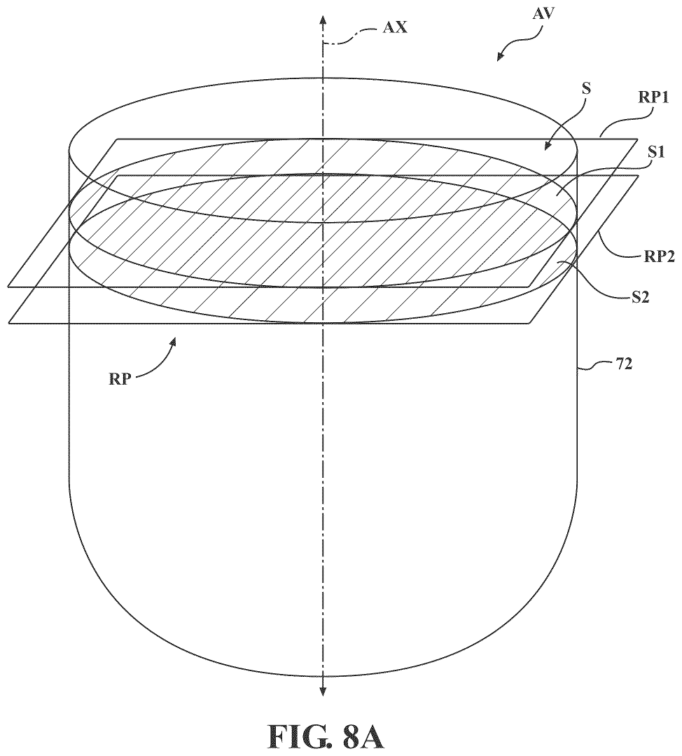

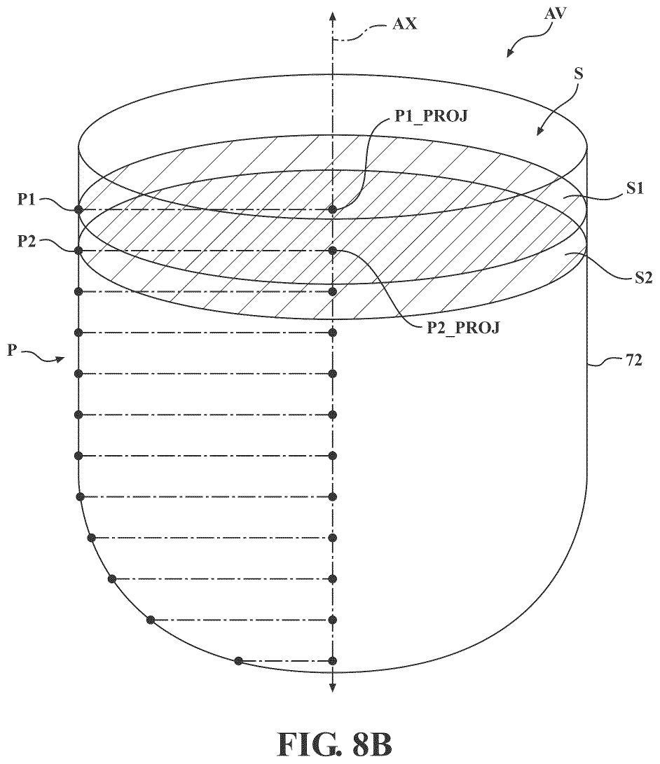

Referring to FIG. 3, the method 1000 includes a step 1008 of generating a plurality of reference planes. As shown in FIG. 8A, the reference planes RP may divide the allowed volume AV. Specifically, the references planes RP may intersect the boundary 72 of the allowed volume AV to divide the allowed volume AV. In the instance of FIG. 8A, a first exemplary reference plane RP1 and a second exemplary reference plane RP2 are shown.

The method 1000 may include a step 1010 of generating a plurality of sections corresponding to the plurality of reference planes RP. An example of the plurality of sections S is shown in FIG. 8A. Specifically, in the instance of FIG. 8A, a first exemplary section S1 corresponding to the first exemplary reference plane RP1 and a second exemplary section S2 corresponding to the second exemplary reference plane RP2 are shown.

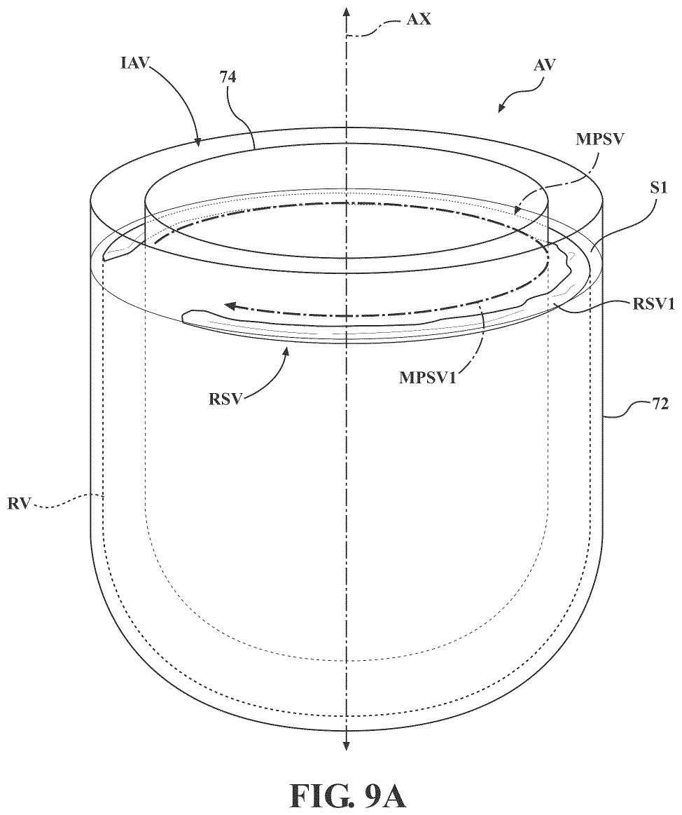

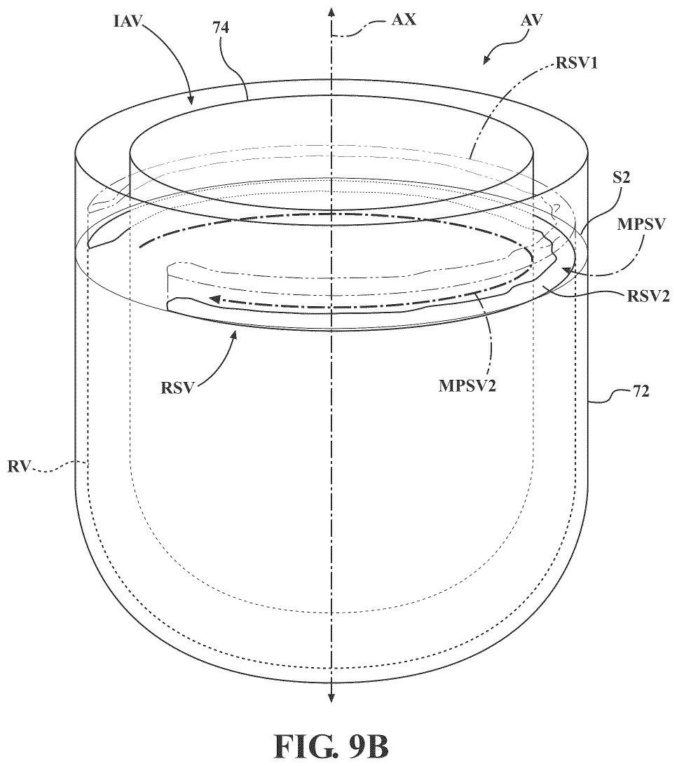

The plurality of sections S are generated to identify resection sub-volumes RSV of the resection volume RV. For example, in FIGS. 9A and 9B, the first exemplary section S1 and the second exemplary section S2 are generated to identify a first exemplary resection sub-volume RSV1 and a second exemplary resection sub-volume RSV2, respectively. In the instance of FIGS. 9A and 9B, the first exemplary section S1 and the second exemplary section S2 are intersected with the resection volume RV to identify the first exemplary resection sub-volume RSV1 and a second exemplary resection sub-volume RSV2, respectively. In other instances, the identified resection sub-volumes RSV may be adjacent to a section S of the plurality of sections S.

The method 1000 generates the milling path MP to resect the resection volume RV by generating a sub-volume milling path MPSV to resect each resection sub-volume RSV. In FIG. 9A, a first sub-volume milling path MPSV1 is generated for resecting the first resection sub-volume RSV1. In FIG. 9B, a second sub-volume milling path MPSV2 is generated for resecting the second resection sub-volume RSV2. Once the end effector 20 has moved along the first sub-volume milling path MPSV1 and resected the first resection sub-volume RSV1, the end effector 20 may proceed to the second sub-volume milling path MPSV2 to resect the second resection sub-volume RSV2. FIGS. 9A and 9B illustrate an example sub-volume resection volumes RSV and an example sub-volume milling paths MPSV. The step of identifying a sub-volume resection volume RSV and the step of generating a sub-volume milling path MPSV will be explained in greater detail herein.

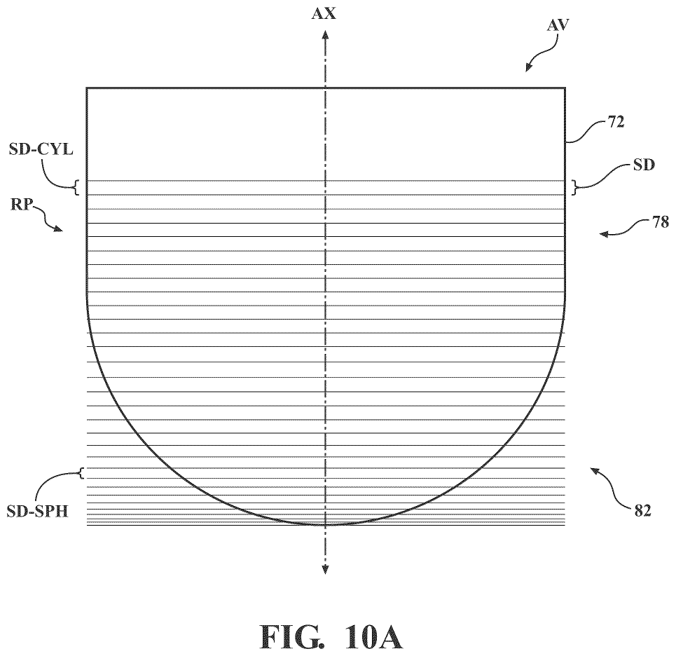

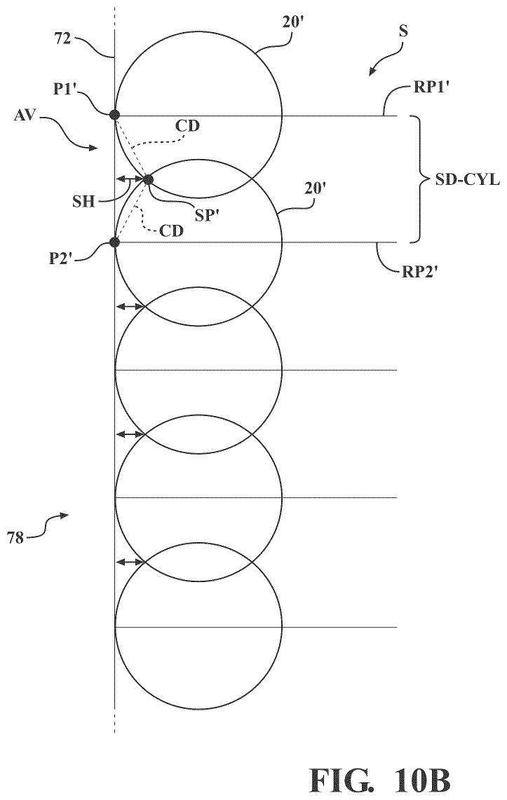

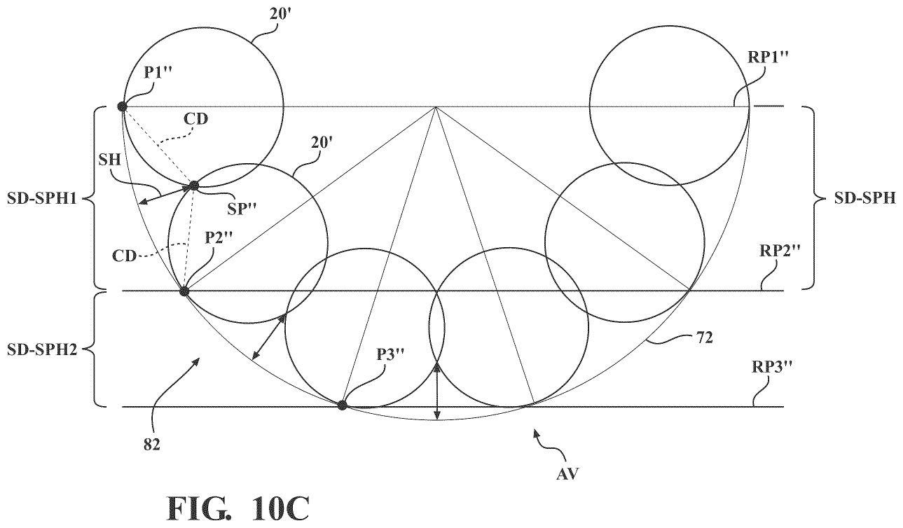

FIG. 10A illustrates an instance of the plurality of reference planes RP. As shown, the plurality of reference planes RP are perpendicular to the axis AX and are parallel to one another. Additionally, adjacent reference planes RP of the plurality of reference planes RP are separated by a step distance SD. In some instances, the step distance SD may be a constant distance. In other instances, the step distance SD may be a variable distance. For example, in the instance of FIG. 10A, the step distance SD is a variable distance that is based on the geometry of the allowed volume AV. For instance, as shown in FIG. 10A, the step distance SD-CYL between adjacent reference planes RP intersecting the cylindrical portion 78 of the allowed volume AV is larger than the step distance SD-SPH between adjacent reference planes RP intersecting the spherical dome portion 82 of the allowed volume AV.

The step distance SD may be calculated based on preserving a desired scallop height SH between adjacent reference planes RP. The desired scallop height SH corresponds to a desired height of hard tissue that remains between adjacent resection sub-volumes RSV after the adjacent resection sub-volumes RSV have been resected. For example, the step distance SD may be calculated such that the desired scallop height SH is a desired minimum acceptable height, a desired maximum acceptable height, and/or a desired acceptable height that allows for the fewest number of reference planes RP.

FIGS. 10B and 10C illustrate an example calculation of the step distance SD based on preserving a desired scallop height SH. In FIG. 10B, the reference planes RP intersecting the cylindrical portion 78 of the allowed volume AV are shown, with a first reference plane RP1′ and a second reference plane RP2′ being identified. The first reference plane RP1′ intersects the boundary 72 of the allowed volume AV at a first intersection point P1′. The second reference plane RP2′ intersects the boundary 72 of the allowed volume AV at a second intersection point P2′. In FIG. 10C, the reference planes RP intersecting the spherical dome portion 82 of the allowed volume AV are shown, with a first reference plane RP1″, a second reference plane RP2″, and a third reference plane RP3″ being identified. The first reference plane RP1″ intersects the boundary 72 of the allowed volume AV at a first intersection point P1″. The second reference plane RP2″ intersects the boundary 72 of the allowed volume AV at a second intersection point P2″. The third reference plane RP3″ intersects the boundary 72 of the allowed volume AV at a third intersection point P3″. As shown, the step distance SD-CYL between adjacent reference planes RP intersecting the cylindrical portion 78 and the step distance SD-SPH between adjacent reference planes RP intersecting the spherical dome portion 82 are both calculated based on preserving a desired scallop height SH.

The scallop height SH is dependent on the geometry of the end effector 20. The scallop height SH is defined, geometrically, as a distance from the boundary 72 of the allowed volume AV to a scallop point SP′, SP″. The location of the scallop point SP′, SP″ is defined in view of the geometry of the end effector 20. To illustrate, representations of the end effector 20′ are superimposed on the allowed volume AV, with the representations of the end effector 20′ being tangent to the boundary 72 at the points of intersection between the reference planes RP and the boundary 72, i.e., intersection points P1′, P1″, P2′, P2″ in FIGS. 10B and 10C. The scallop points SP′, SP″ are defined as being located at an intersection of adjacent representations of the end effector 20′, where the scallop points SP′, SP″ are located at a common distance CD from the first intersection point P1′, P1″ and the second intersection point P2′, P2″. As should be understood from the above description, should a geometry of the end effector 20 vary from the geometry shown in FIGS. 10B and 10C, the step distance SD between the plurality of reference planes RP would also vary.

The step distance SD-CYL between adjacent reference planes RP intersecting the cylindrical portion 78 and the step distance SD-SPH between adjacent reference planes RP intersecting the spherical dome portion 82 are calculated to preserve the desired scallop height SH. In FIG. 10B, the step distance SD-CYL is calculated to be the same between any two reference planes RP to preserve the desired scallop height SH. In FIG. 10C, the step distance SD-SPH is calculated to be the variable between reference planes RP to preserve the desired scallop height SH. For example, a step distance SD-SPH1 between reference planes RP1″, S2″ is greater than a step distance SD-SPH2 between reference planes RP2″, RP3″.

The desired scallop height SH may be selected based on a desired height of hard tissue that remains between adjacent resection sub-volumes RSV after the adjacent resection sub-volumes RSV have been resected. Additionally, the desired scallop height SH is defined, geometrically, as a distance from the boundary 72 of the allowed volume AV to a scallop point SP′, SP″. As such, in instances where the desired height of hard tissue that remains between adjacent resection sub-volumes RSV is greater than the scallop height SH of FIGS. 10B and 10C, a location of the representations of the end effector 20′ may be spaced further apart such that the scallop points SP′, SP″ are further from the boundary 72 of the allowed volume AV. As follows, because the representations of the end effector 20′ are defined as being tangent to the boundary 72 at the points of intersection between the reference planes RP and the boundary 72, the reference planes RP may also be spaced further apart, and the step distance SD may be greater. Similarly, in instances where the desired height of hard tissue that remains between adjacent resection sub-volumes RSV is less than the scallop height SH of FIGS. 10B and 10C, a location of the representations of the end effector 20′ may be closer to one another such that the scallop points SP′, SP″ are closer to the boundary 72 of the allowed volume AV. As follows, the reference planes RP may also be closer to one another, and the step distance SD may be smaller.

Accordingly, as the adjacent reference planes RP may be separated by a step distance SD, corresponding adjacent sections S of the plurality of sections S may also be separated by a step distance SD. For example, in instances where the sections S are coplanar with a corresponding reference plane RP, such as the instance of FIG. 8A, adjacent sections S may also be separated by the step distance SD. As another example, in instances where one or more of the adjacent sections S include a nonplanar geometry, a point on the first section S may be separated from a point on the adjacent section S by the step distance SD.

In various instances of the method, aspects of the plurality of reference planes RP may vary. In some instances, an orientation of the reference planes RP relative to the axis AX may vary. In the instance of FIG. 10A, the plurality reference planes RP are oriented such that the reference planes RP are perpendicular to the axis AX. In other instances, however, the plurality of reference planes RP may be oriented such that the reference planes RP are not perpendicular to the axis AX. The plurality of reference planes RP may also be oriented such that the plurality of reference planes RP are not parallel to one another. In some instances, a distance between the reference planes RP may vary. As shown in FIG. 10A, adjacent reference planes RP are separated by a step distance SD. In some instances, the step distance SD may be a constant distance. In other instances, the step distance SD may be a variable distance. For example, in the instance of FIG. 10A, the step distance SD is a variable distance that is based on the geometry of the allowed volume AV. For instance, as shown in FIG. 10A, the step distance SD-CYL between reference planes RP adjacent to the cylindrical portion 78 of the allowed volume AV is larger than the step distance SD-SPH between reference planes RP adjacent to the spherical dome portion 82 of the allowed volume AV.

In some instances, the method 1000 may optionally omit generation of the plurality of references planes RP. For example, in the instance of FIG. 8B, the method 1000 instead generates a plurality of points P along the boundary 72 of the allowed volume AV, such as a first exemplary point P1 and a second exemplary point P2. In the instance of FIG. 8B, the points P are generated along the boundary 72 such that a projection P_PROJ of each of the points P along the axis AX has a unique location. As an example, a first projected point P1_PROJ corresponds to the first exemplary point P1 and is defined as a projection of the first exemplary point P1 along the axis AX; and a second projected point P2_PROJ corresponds to the second exemplary point P2 and is defined as a projection of the second exemplary point P2 along the axis AX. As shown, a location along the axis AX of the projected point P1_PROJ is different from a location along the axis AX of the second projected point P2_PROJ. In such an instance, each of the plurality of points P correspond to a section S of the plurality of sections S. For example, the first exemplary point P1 corresponds to a first exemplary section S1, and a second exemplary point P2 corresponds to a second exemplary section S2.

v. Generating Plurality of Sections Corresponding to Reference Planes

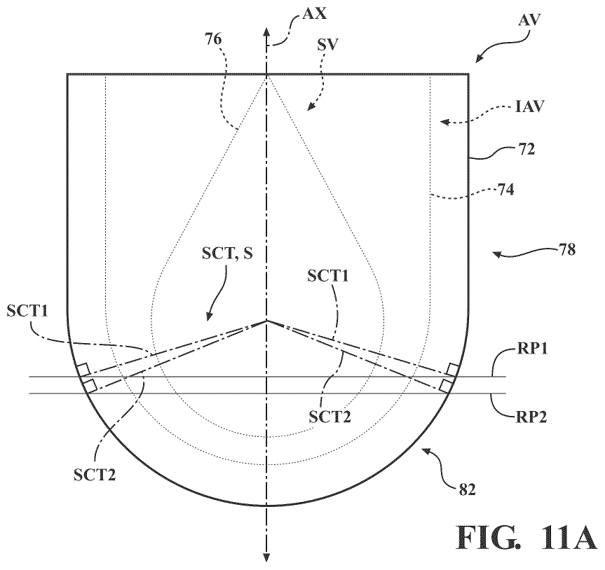

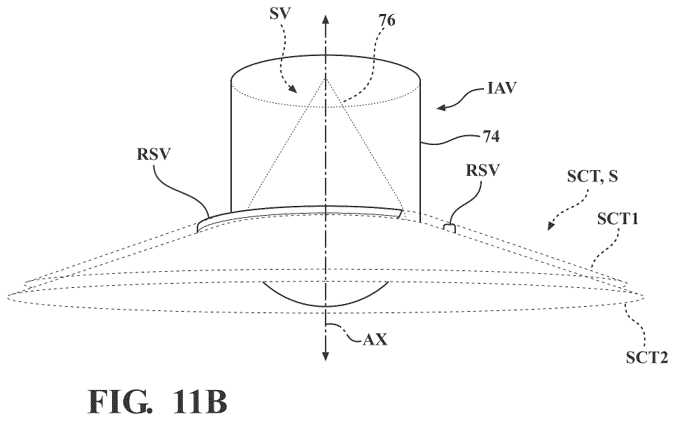

As previously discussed, the method 1000 includes the step 1010 of generating a plurality of sections S corresponding to the plurality of reference planes RP. The plurality of sections S may include any suitable geometry. For example, of the plurality of sections S shown in FIG. 8A include a planar geometry. An additional example of the plurality of sections S is shown in FIGS. 11A and 11B, where the plurality of sections S include a non-planar geometry. In such instances, the plurality of sections S may be further defined as a plurality of sectors SCT, the plurality of sector SCT including a non-planar geometry. The plurality of sections S may include a combination of sections S including a planar geometry and sectors SCT including a non-planar geometry. For instance, the plurality of sections S may include only sections S including a planar geometry or at least one sector SCT including a non-planar geometry. Herein, in instances where the section S includes a non-planar geometry, the section S may be referred to as a sector SCT.

In the instance of FIGS. 11A and 11B, the method 1000 generates, during step 1010, a plurality of sectors SCT corresponding to each reference plane RP during step 1010. A sector SCT is defined as extending radially from the axis AX toward the boundary 72 of the allowed volume AV, where the sector SCT is defined as being normal to the boundary 72 at an intersection of a reference plane RP and the boundary 72. For example, referring to FIG. 11A, a first sector SCT1 normal to the boundary at an intersection of the first reference plane RP1 and the boundary 72 is generated, and a second sector SCT2 normal to the boundary 72 at an intersection of the second reference plane RP2 and the boundary 72 is generated. In the instance of FIG. 11A, the first and sector SCT1, SCT2 are shown two-dimensionally to illustrate an angular relationship between the first and second sector SCT1, SCT2 and the boundary 72. Referring to FIG. 11B, the first sector SCT1 and the second sector SCT2 are shown as three-dimensional objects.

vi. Generating Sub-Volume Milling Paths

Steps 1012-1020 shown in FIG. 3 are for generating a sub-volume milling path MPSV for a single section S. As previously stated, the method 1000 generates the milling path MP to resect the resection volume RV by generating a sub-volume milling path MPSV to resect a resection sub-volume RSV corresponding to the section S. Steps 1012-1020 may be repeated and a sub-volume milling path MPSV may be generated for any suitable number of sections S, such as for each section S of the plurality of sections S, for every other section S of the plurality of sections S, or for more than one section S of the plurality of sections S. In instances where the several sub-volume milling paths MPSV are generated, the sub-volume milling paths MPSV together form the milling path MP. In instances where a single sub-volume milling path MPSV is generated, the single sub-volume milling paths MPSV is the milling path MP.

a. Identifying the Resection Sub-Volume

Referring to FIG. 3, the method 1000 identifies the resection sub-volume RSV of the resection volume RV corresponding to a section S during step 1012. FIGS. 11A and 11B illustrate an instance where a resection sub-volume RSV adjacent to the spherical dome portion 82 of the allowed volume AV is identified. In the instance of FIGS. 11A and 11B, the sections S are further defined as sectors SCT including a non-planar geometry. The resection sub-volume RSV is identified in FIG. 11B as a portion of the resection volume RV between the first sector SCT1 and the second sector SCT2, with the identified resection sub-volume RSV corresponding to either the first sector SCT1 or the second sector SCT2.

In the instance of FIGS. 11A and 11B, a resection sub-volume RSV adjacent to the spherical dome portion 82 of the allowed volume AV is identified. In other instances, step 1012, as described above, may be used to identify a resection sub-volume RSV adjacent to any other portion of the allowed volume AV, such as the cylindrical portion 78. Additionally, step 1012, as described above, may be used to identify a resection sub-volume RSV adjacent to a portion of an allowed volume AV including a different geometry.

In some instances, the method 1000 may identify the resection sub-volume RSV without generating two adjacent sections S or sectors SCT. For example, in some instances, the method 1000 may identify, during step 1012, the resection sub-volume RSV as the sub-volume of the resection volume RV between a single sector SCT and a step distance SD above or below the single sector SCT. As another example, in some instances, the method 1000 may identify, during step 1012, the resection sub-volume RSV as the sub-volume of the resection volume RV between a half step distance SD above a single sector SCT and a half step distance SD below the single sector SCT.

b. Generating Milling Path Segments

Referring to FIG. 3, the method 1000 includes a step of generating one or more milling path segments MPS designed to enable the end effector 20 to remove the resection sub-volume RSV of the resection volume RV. A milling path segment MPS may be defined as a portion of a sub-volume milling path MPSV along which the end effector 20 moves to resect the resection sub-volume RSV.

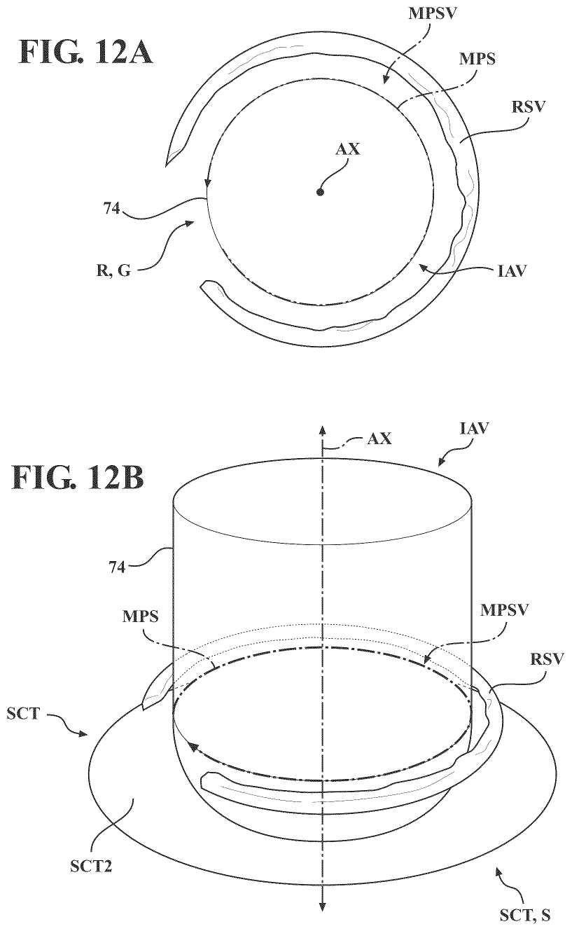

An example milling path segment MPS is shown in FIGS. 12A and 12B. As shown in FIGS. 12A and 12B, the milling path segment MPS is generated such that, when the end effector 20 moves along the milling path segment MPS, the end effector 20 resects the resection sub-volume RSV. The milling path segment MPS is generated based on the geometry of the inner allowed volume IAV and based on an intersection of the inner allowed volume IAV and a section S. FIG. 12A illustrates a top-down view of the resection sub-volume RSV and the inner allowed volume IAV to illustrate that the milling path segment MPS is generated based on the geometry of the inner allowed volume IAV. The inner allowed volume IAV includes a circular cross-sectional shape and, accordingly, the milling path segment MPS also includes a circular shape. Additionally, FIG. 12A illustrates that the milling path segment MPS is generated along the boundary 74 of the inner allowed volume IAV. Further shown in FIG. 12B, the milling path segment MPS is generated along the boundary 74 at the intersection of the boundary 74 and the second sector SCT2. In an alternative instance, the milling path segment MPS may be generated at the intersection of the boundary 74 of the inner allowed volume IAV and the first sector SCT1.

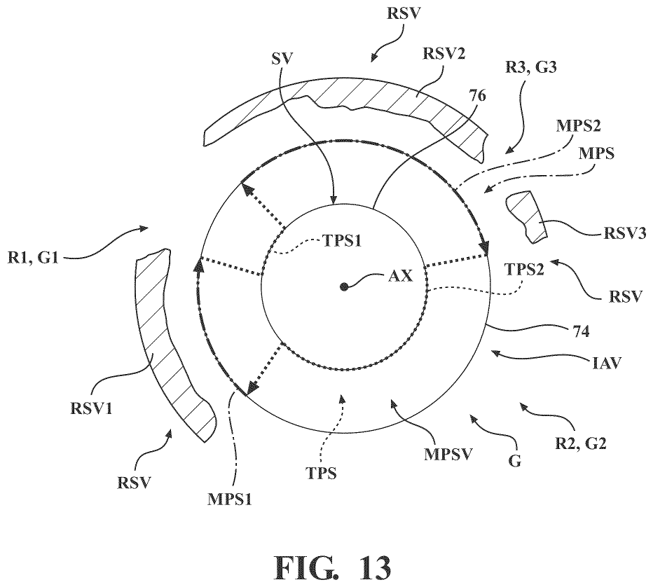

In the instance of FIGS. 12A and 12B, a single milling path segment MPS is generated to resect the resection sub-volume RSV. In such instances, the sub-volume milling path MPSV includes the single milling path segment MPS. However, in other instances, more than one milling path segment MPS may be generated to resect the resection sub-volume RSV. In the instance of FIG. 13, the sub-volume milling path MPSV includes a first milling path segment MPS1 for resecting a first portion of the resection sub-volume RSV1 and a second milling path segment MPS2 for resecting a second and third portion of the resection sub-volume RSV2, RSV3. In other instances, any suitable number of milling path segments MPS may be generated to resect the resection sub-volume RSV.

c. Generating Transition Path Segments

The method 1000 also includes a step 1016 of identifying, for the resection sub-volume RSV, a region to be avoided by the end effector 20 and a step 1018 of generating one or more transition path segments TPS designed to avoid the region. As previously stated, the end effector 20 moves along transition path segments TPS to avoid resection of soft tissue. A transition path segment TPS may be defined as a portion of a sub-volume milling path MPSV along which the end effector 20 moves to avoid resection of soft tissue.

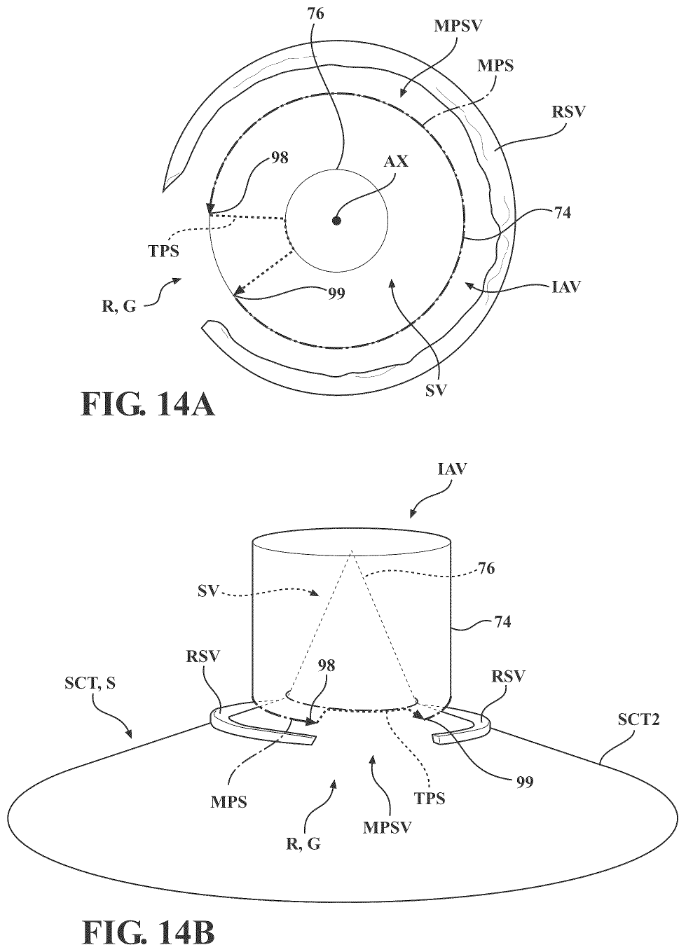

As previously stated, soft tissue may be omitted from the model 70. For example, soft tissue may exist in areas where the model 70 does not indicate the presence of hard tissue. In other words, there is a possibility that soft tissue exists in regions where there exists an absence of the resection volume RV. Regions where soft tissue may exist may be identified as regions to be avoided by the end effector 20 and the transition path segments TPS may be generated to avoid the regions. As such, during step 1016, the method 1000 may identify a region R to be avoided by the end effector 20 by identifying that the resection sub-volume RSV is interrupted by a gap G defining an absence of the resection volume RV, wherein the region R to be avoided by the end effector 20 includes the gap G. For example, referring to the instance of FIGS. 14A and 14B, the region R is a region to be avoided by the end effector 20 as the region R includes a gap G and the gap G interrupts the resection sub-volume RSV.

During step 1018, the method 1000 generates one or more transition path segments TPS designed to avoid a region to be avoided by the end effector 20. For example, referring to the instance of FIGS. 14A and 14B, the method 1000 generates the transition path segment TPS to avoid the region R, which includes the gap G. Additionally, the method 1000 generates the one or more transition path segments TPS such that the one or more transition path segments TPS connect the one or more milling path segments MPS for the section S. As shown in FIGS. 14A and 14B, the transition path segment TPS connects a first end 98 and a second end 99 of the milling path segment MPS.

The one or more transition path segments TPS may be generated based on the geometry of the inner allowed volume IAV and extends along the boundary 76 of the safeguard volume SV. FIG. 14A illustrates a top-down view of the resection sub-volume RSV and the inner allowed volume IAV to illustrate that the transition path segment TPS is generated based on the geometry of the safeguard volume SV. The safeguard volume SV includes a circular cross-sectional shape and, accordingly, the transition path segment TPS includes a portion extending along the boundary 76, where the transition path segment TPS also includes a circular shape. The one or more transition path segments TPS may also be generated to extend along the sector SCT between the one or more milling path segments MPS and the boundary 76 of the safeguard volume SV. FIG. 14B illustrates that the transition path segment TPS includes a portion extending along the second sector SCT2 between the milling path segment MPS and the boundary 76. In an alternative instance, the transition path segment TPS may be generated to extend along the first sector SCT1 between the milling path segment MPS and the boundary 76.

In the instance of FIGS. 14A and 14B, a single transition path segment TPS is generated to avoid resect the region R. In such instances, the sub-volume milling path MPSV includes the single transition path segment TPS. However, in other instances, more than one transition path segment TPS may be generated to avoid more than one region R. In the instance of FIG. 13, the sub-volume milling path MPSV includes a first transition path segment TPS1 for avoiding a first region R1 including a first gap G1, and a second transition path segment TPS2 for avoiding a second region R2 including a second gap G2. In other instances, any suitable number of milling path segments MPS may be generated to avoid any number of regions R.

In some instances, the method 1000 may optionally omit generation of a transition path segment TPS to avoid a region R including a gap G.

In one such instance, the method 1000 may include a step of determining whether a dimension of a gap G is greater than a threshold value before generating a transition path segment TPS to avoid a region R including the gap G. The threshold value may be selected based on a possibility that soft tissue exists in a region R where there exists an absence of the resection volume RV. For example, in an instance where a region R includes a gap G, the threshold value may be a predetermined gap threshold size and the predetermined gap threshold size may be selected such that there is a high possibility that soft tissue exists in the region R should a size of a gap G be greater than the predetermined gap threshold size. Similarly, the predetermined gap threshold size may be selected such that there is a low possibility that soft tissue exists in the region R should a size of a gap G be less than the predetermined gap threshold size.

In another such instance, the method 1000 may optionally omit generation of a transition path segment TPS to avoid a region R including a gap G if the resection sub-volume is interrupted by a single gap. For example, referring to FIG. 12A, the resection sub-volume RSV is interrupted by a single gap G. In such an instance, the method 1000 may optionally omit generation of a transition path segment TPS. Instead, the method 1000 may proceed directly to a different sub-volume milling path MPSV corresponding to a different section S once the end effector 20 has moved along the milling path segment MPS.

FIG. 13 illustrates an instance where the method 1000 omits generation of a transition path segment TPS in response to determining that a gap G of a region R is smaller than a predetermined gap threshold size. As shown, the first gap G1 of the first region R1 may be defined as an area between the first and second portions of the resection sub-volume RSV1, RSV2, the second gap G2 of the second region R2 may be defined as an area between the third and first portions of the resection sub-volume RSV3, RSV1, and a third gap G3 of a third region R3 may be defined as an area between the second and third portions of the resection sub-volume RSV2, RSV3. As shown, a size of the first and second gap G1, G2 is larger than a size of the third gap G3. In the instance of FIG. 13, the method 1000 determines that the first gap G1 is greater than the predetermined gap threshold size and, therefore, generates a first transition path segment TPS1 to avoid the first region R1. The method 1000 also determines that the second gap G2 is greater than the predetermined gap threshold size and, therefore, generates a second transition path segment TPS2 to avoid the second region R2. However, the method 1000 determines that the third gap G3 is not greater than the predetermined gap threshold size and, therefore, does not generate a transition path segment TPS to avoid the third region R3.

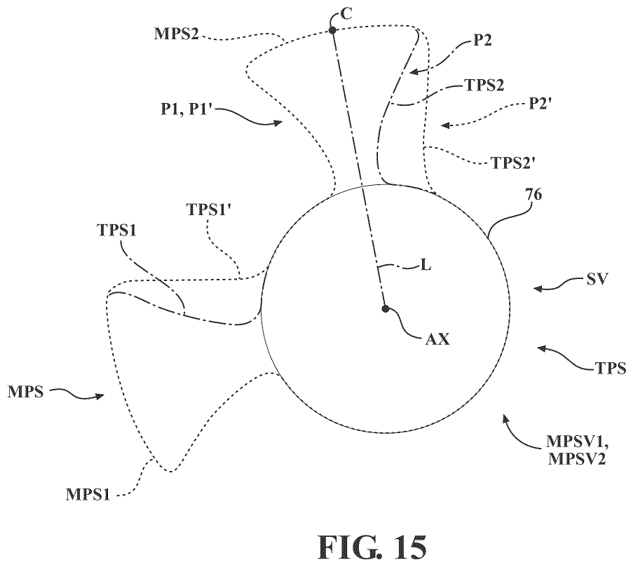

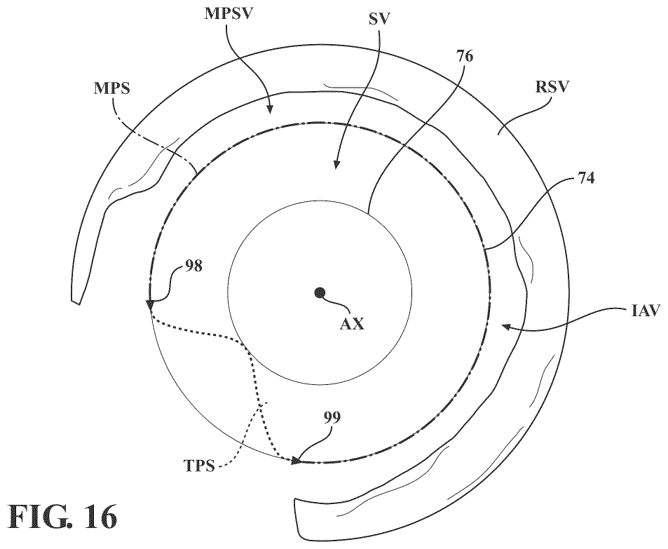

In the instance of FIGS. 13, 14A, and 14B, the transition path segments TPS are generated such that the transition path segments TPS connect one or more milling path segments MPS by sharply proceeding from a milling path segment MPS toward the boundary 76 of the safeguard volume SV. In other instances, such as the instances of FIGS. 15 and 16, the transition path segments TPS are generated to connect one or more milling path segments MPS by smoothly proceeding from a milling path segment MPS toward the boundary 76 of the safeguard volume SV.

Transition path segments TPS of a sub-volume milling path MPSV may be generated to proceed symmetrically and asymmetrically, respectively, between the boundary 76 and a milling path segment MPS of the sub-volume milling path MPSV. As shown in FIG. 15, a first sub-volume milling path MPSV1 includes a first and second milling path MPS1, MPS2 and a first and second transition path segments TPS1, TPS2; and a second sub-volume milling path MPSV2 includes the first and second milling path MPS1, MPS2 and first and second transition path segments TPS1′, TPS2′. For the first sub-volume milling path MPSV1, a portion P1 of the first transition path segment TPS1 that proceeds from the boundary 76 toward the second milling path MPS2 is symmetric to a portion P2 of the second transition path segment TPS2 that proceeds from the second milling path MPS2 toward the boundary 76, where is symmetry is defined in relation to a line L that extends from the axis AX to a center C of the second milling path MPS2. For the second sub-volume milling path MPSV2, a portion P1′ of the first transition path segment TPS1′ that proceeds from the boundary 76 toward the second milling path MPS2 is asymmetric to a portion P2′ of the second transition path segment TPS2′ that proceeds from the second milling path MPS2 toward the boundary 76, where is symmetry is defined in relation to a line L that extends from the axis AX to a center C of the second milling path MPS2. In some instances, it may be advantageous for the transition path segments TPS to proceed symmetrically or asymmetrically between the boundary 76 and a milling path segment MPS. For instance, it may be advantageous for the transition path segments TPS to proceed symmetrically to avoid resecting soft tissue. It may be advantageous for the transition path segments TPS to proceed asymmetrically to optimize the transition path segments TPS, as will be described in greater detail below.

Transition path segments TPS of a sub-volume milling path MPSV may be optimized based on several parameters to minimize a cutting time of the end effector 20. For example, the transition path segment TPS may be optimized to minimize a distance traveled by the end effector 20 and the transition path segment TPS may be optimized for maximum smoothness. Such optimization may be useful to reduce time spent air cutting by the end effector 20, when the end effector 20 does not resect material. Additionally, such optimization reduces a number of sharp turns the end effector 20 makes while traveling along a sub-volume milling path MPSV. As sharp turns may leave a mark on the bone, such optimization allows the end effector 20 to smoothly resect the resection sub-volume RSV.

For example, in the instance of FIG. 15, the transition path segment TPS is optimized. Specifically, as the transition path segments TPS1′, TPS2′ proceed asymmetrically, a distance traveled by the end effector 20 when traveling along the transition path segments TPS1′, TPS2′ is minimized.

As another example, the transition path segment TPS in the instance of FIG. 16 is optimized. Referring to FIG. 16, the sub-volume milling path MPSV includes a transition path segment TPS that connects a first end 98 and a second end 99 of the milling path segment MPS. In the instance of FIG. 16, the transition path segment TPS is optimized such that a distance traveled by the end effector 20 when traveling along the transition path segment TPS is minimized, and the transition path segment TPS is optimized such that a smoothness of the transition path segment TPS is maximized.

d. Combining Milling Path Segments and Transition Path Segments

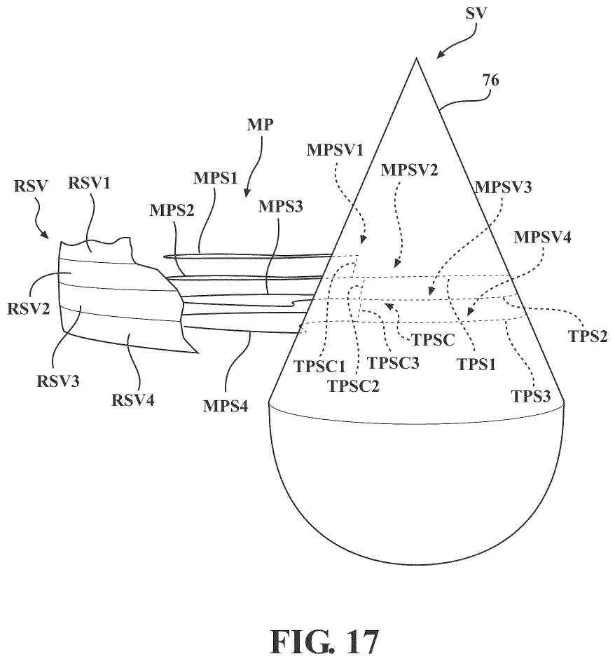

Referring to FIG. 3, the method 1000 includes a step 1020 of generating the sub-volume milling path MPSV by combining the one or more milling path segments MPS and the one or more transition path segments TPS. For example, referring to FIG. 13, the first and second milling path segments MPS1, MPS2 and the first and second transition path segments TPS1, TPS2 are combined to form the sub-volume milling path MPSV. As another example, referring to FIG. 17, a first milling path segment MPSV1 includes a first milling path segment MPS1, a second milling path segment MPSV2 includes a second milling path segment MPS2 and a first transition path segment TPS1, a third milling path segment MPSV3 includes a third milling path segment MPS3 and a second transition path segment TPS2, and a fourth milling path segment MPSV4 includes a fourth milling path segment MPS4 and a third transition path segment TPS3.

As previously stated, the method 1000 generates the milling path MP to resect the resection volume RV by generating a sub-volume milling path MPSV to resect each resection sub-volume RSV. For example, referring to FIG. 17, the milling path MP includes the first sub-volume milling path MPSV1 for resecting a first resection sub-volume RSV1, the second sub-volume milling path MPSV2 for resecting a second resection sub-volume RSV2, the third sub-volume milling path MPSV3 for resecting a third resection sub-volume RSV3, and the fourth sub-volume milling path MPSV4 for resecting a fourth resection sub-volume RSV4.

In some instances, the method 1000 may include a step of generating a connecting transition path segment TPSC for connecting sub-volume milling paths MPSV. The one or more connecting transition path segments TPSC allows the end effector 20 to move from a sub-volume milling path MPSV of a first section S to a sub-volume milling path MPSV of an adjacent second section S. For example, referring to FIG. 17, the first connecting transition path segment TPSC1 connects the first milling path segment MPS1 of the first sub-volume milling path MPSV1 to the second milling path segment MPS2 of the second sub-volume milling path MPSV2; the second connecting transition path segment TPSC2 connects the second milling path segment MPS2 of the second sub-volume milling path MPSV2 to the third milling path segment MPS3 of the third sub-volume milling path MPSV3; and the third connecting transition path segment TPSC3 connects the third milling path segment MPS3 of the third sub-volume milling path MPSV3 to the fourth milling path segment MPS4 of the third sub-volume milling path MPSV4. While the connection transition path segments TPSC of FIG. 17 are generated between milling path segments MPS, in other instances, the connection transition path segments TPSC may be generated between transition path segments TPS, or between a transition path segment TPS and a milling path segment MPS.

The connecting transition path segments TPSC extend along the boundary 76 of the safeguard volume between adjacent sections S. As shown in FIG. 17, the first connecting transition segment TPSC1 extends along the boundary 76 between the first sub-volume milling path MPSV1 and the second sub-volume milling path MPSV2, the second connecting transition segment TPSC2 extends along the boundary 76 between the second sub-volume milling path MPSV2 and the third sub-volume milling path MPSV3, and the third connecting transition segment TPSC3 extends along the boundary 76 between the third sub-volume milling path MPSV3 and the fourth sub-volume milling path MPSV4.

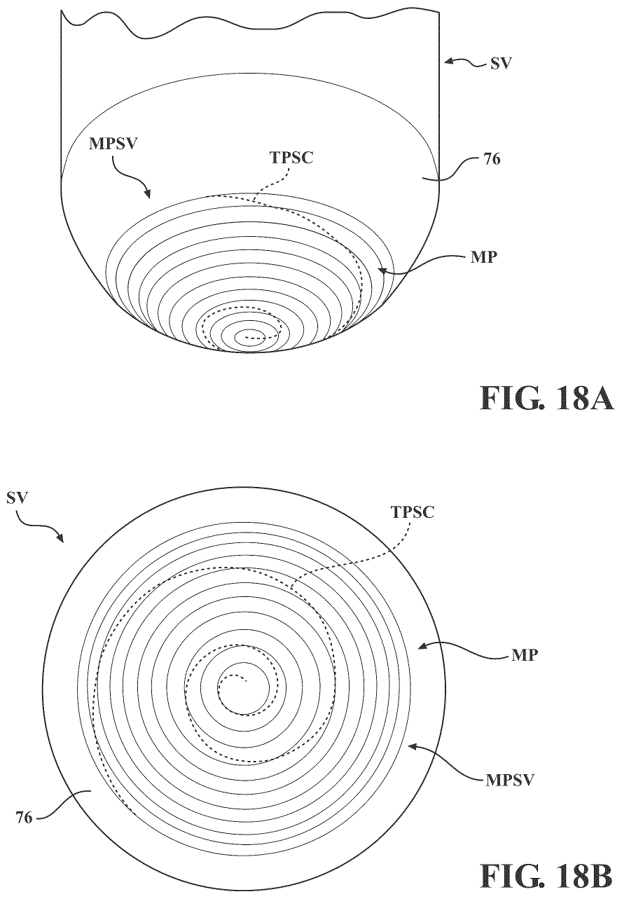

In some instances, the connecting transition path segments TPSC may combine to form a spiral. For example, referring to FIGS. 18A and 18B, the connecting transition path segments TPSC connect sub-volume milling paths MPSV of the milling path MP, extend along the boundary 76, and combine to form a spiral between the sub-volume milling paths MPSV. A spiral shape of the connecting transition path segments TPSC may be useful in instances where the connecting transition path segments TPSC connect sub-volume milling paths MPSV that do not include transition path segments TPS. In such instances, the resection sub-volumes RSV to be resected by the sub-volume milling paths MPSV may not be interrupted by gaps. By forming a spiral shape, the connecting transition path segments TPSC optimize a path of the end effector 20 as the end effector 20 travels from one sub-volume milling path MPSV to another, and aids in complete resection of the resection sub-volumes RSV. For instance, the spiral shape allows the end effector 20 to travel less distance as the end effector 20 moves from one sub-volume milling path MPSV to another. As follows, the spiral shape allows the end effector 20 to resect the resection volume RV in less time. Furthermore, the spiral shape reduces a number of sharp turns the end effector 20 makes while traveling from one sub-volume milling path MPSV to another. As sharp turns may leave a mark on the bone, the spiral shape allows the end effector 20 to smoothly resect the resection volume RV.

While connecting transition path segments TPSC are described herein as combining to form a spiral, in other instances, the connecting transition path segments TPSC may form any other suitable shape. For example, the connecting transition path segments TPSC may form any other shape suitable for optimizing a path of the end effector 20 and for aiding in complete resection of the resection volume RV.

Although specific features of various embodiments of the disclosure may be shown in some drawings and not in others, this is for convenience only. In accordance with the principles of the disclosure, any feature of a drawing or other embodiment may be referenced and/or claimed in combination with any feature of any other drawing or embodiment.

This written description uses examples to describe embodiments of the disclosure and also to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computer-implemented method for generating a milling path for a tool of a surgical system, the milling path designed to enable the tool to resect material from a bone that defines a socket for a joint, the computer-implemented method comprising:
obtaining a model of the bone including the socket;
intersecting an allowed volume with the model of the socket for defining a resection volume intended to be removed from the bone;
generating a plurality of sections;
for at least one section:
identifying a sub-volume of the resection volume corresponding to the section;
generating one or more milling path segments designed to enable the tool to remove the sub-volume of the resection volume;
identifying, for the sub-volume of the resection volume, a region to be avoided by the tool;
generating one or more transition path segments designed to avoid the region; and
generating the milling path by combining the one or more milling path segments and the one or more transition path segments.

2. The computer-implemented method of claim 1, further comprising generating a safeguard volume within the allowed volume, wherein the safeguard volume defines a boundary for the tool, wherein the allowed volume defines a boundary for the tool, wherein the boundary of the safeguard volume is spaced apart from the boundary of the allowed volume, and wherein the one or more transition path segments are defined by a geometry of the safeguard volume.

3. The computer-implemented method of claim 1, further comprising identifying that the sub-volume of the resection volume is interrupted by a gap defining an absence of the resection volume, wherein the region to be avoided by the tool includes the gap.

4. The computer-implemented method of claim 3, further comprising determining whether a dimension of the gap is greater than a threshold value and generating the one or more transition path segments in response to determining that the dimension of the gap is greater than the threshold value.

5. The computer-implemented method of claim 2, further comprising generating an inner allowed volume within the allowed volume, the inner allowed volume defining a boundary for the tool, wherein the boundary of the inner allowed volume is spaced apart from the boundary of the allowed volume, and wherein the safeguard volume is defined within the inner allowed volume and the boundary of the safeguard volume is spaced apart from the inner allowed volume.

6. The computer-implemented method of claim 5, wherein the tool includes a spherical cutting burr having a burr radius, and wherein the boundary of the inner allowed volume is spaced apart from the boundary of the allowed volume by the burr radius.

7. The computer-implemented method of claim 5, wherein the allowed volume extends along an axis.

8. The computer-implemented method of claim 7, wherein the allowed volume, the inner allowed volume, and the safeguard volume are coaxial about the axis.

9. The computer-implemented method of claim 7, wherein the allowed volume, the inner allowed volume, and the safeguard volume are each rotationally symmetric.

10. The computer-implemented method of claim 7, wherein the section is further defined as a sector extending radially from the axis toward the boundary of the allowed volume.

11. The computer-implemented method of claim 10, wherein the sector is defined as being normal to the boundary of the allowed volume.

12. The computer-implemented method of claim 10, wherein the sector is further defined as a first sector, wherein a section adjacent to the section is further defined as a second sector, and wherein identifying the sub-volume of the resection volume corresponding to the first sector comprises identifying the sub-volume of the resection volume between the first and second sectors.

13. The computer-implemented method of claim 5, wherein the one or more milling path segments are generated based on an intersection of the inner allowed volume and the section.

14. The computer-implemented method of claim 5, wherein generating the one or more transition path segments includes generating one or more transition path segments to connect the one or more milling path segments for the at least one section, and wherein the one or more transition path segments extend along the section between the one or more milling path segments and the boundary of the safeguard volume.

15. The computer-implemented method of claim 2, comprising generating a connecting transition path segment for connecting a milling path segment of a first section and a milling path segment of an adjacent second section, wherein the connecting transition path segment extends along the boundary of the safeguard volume between the first section and the adjacent second section.

16. The computer-implemented method of claim 15, wherein the plurality of connecting transition path segments form a spiral extending along the boundary of the safeguard volume.

17. The computer-implemented method of claim 1, wherein a geometry of the allowed volume is based on a geometry of an implant to be inserted into the socket.

18. The computer-implemented method of claim 7, wherein the allowed volume includes a cylindrical portion including a first end and a second end along the axis, the first end and the second end defining a height of the cylindrical portion.

19. The computer-implemented method of claim 18, wherein the allowed volume includes a spherical dome portion having a center located on the axis, and wherein the spherical dome portion is integrated with the cylindrical portion and extends from the second end of the cylindrical portion.

20. The computer-implemented method of claim 1, wherein the bone is a pelvis or a scapula.

21. A non-transitory computer readable medium comprising instructions executable by one or more processors, wherein the instructions implement a software program for generating a milling path for a tool of a surgical system, the milling path designed to enable the tool to resect material from a bone that defines a socket for a joint, the software program being configured to:
obtain a model of the bone including the socket;
intersect an allowed volume with the model of the socket to define a resection volume intended to be removed from the bone;
generate a plurality of sections;
for at least one section:
identify a sub-volume of the resection volume corresponding to the section;
generate one or more milling path segments designed to enable the tool to remove the sub-volume of the resection volume;
identify, for the sub-volume of the resection volume, a region to be avoided by the tool;
generate one or more transition path segments designed to avoid the region; and
generate the milling path by combining the one or more milling path segments and the one or more transition path segments.

22. A surgical system comprising:
a manipulator comprising a robotic arm formed of a plurality of links and joints and supporting a tool;
a control system configured to generate a milling path designed to enable the tool to resect material from a bone that defines a socket for a joint, wherein to generate the milling path, the control system is configured to:
obtain a model of the bone including the socket;
intersect an allowed volume with the model of the socket to define a resection volume intended to be removed from the bone;
generate a plurality of sections; and
for at least one section:
identify a sub-volume of the resection volume corresponding to the section;
generate one or more milling path segments designed to enable the tool to remove the sub-volume of the resection volume;
identify, for the sub-volume of the resection volume, a region to be avoided by the tool; and
generate one or more transition path segments designed to avoid the region; and
generate the milling path by combining the one or more milling path segments and the one or more transition path segments;
wherein the control system is configured to control the manipulator to move the tool along the generated milling path.
